(19) United States
(12) Patent Application Publication
(10) Pub. No.: US 2005/0003000 A1
(43) Pub. Date: Jan. 6, 2005

(54) Method for the the formation of ibuprofen crystals

(76) Inventors: Heinz Einig, Neustadt (DE); Bernd W. Muller, Flintbek (DE); Norbert Rasenack, Kiel (DE); Katrin Friese, Mannheim (DE); Dirk Franke, Birkenheide (DE)

Correspondence Address: KEIL & WEINKAUF, 1350 CONNECTICUT AVENUE, N.W., WASHINGTON, DC, 20036, US

(21) Appl. No.: 10/494,764

(22) PCT Filed: Oct. 25, 2002
(86) PCT No.: PCT/EP02/11999

(30) Foreign Application Priority Data
Nov. 6, 2001 (DE) 10153934.7

(51) Int. Cl. A61K 9/48; A61K 9/26; A61K 9/16; A61K 9/50

(52) U.S. Cl. 424/451; 424/497; 424/469

(57) ABSTRACT

The present invention relates to a process for the formation of profen crystals, which comprises carrying out the formation of solids by displacement precipitation, cooling crystallization, evaporative crystallization or a combination thereof in the presence of one or more additives, and the use of the profens thus prepared for pharmaceutical formulations.

The present invention relates to a process for the formation of profen crystals, and to the use of the profens thus prepared for pharmaceutical administration forms.

As hydrophobic acidic active compounds, the analgesics of the group consisting of the profens are poorly water-soluble substances. This applies in particular in weakly acidic and acidic pH ranges. Thus the low rate of dissolution is the bioavailability-limiting step.

Profens have poor flow properties (strongly cohesive behavior) and poor tabletability (strong adhesion to the die tools and poor plastic deformability). These properties lead to tablets or pressings having low strengths, such that for the equalization of the unsatisfactory pharmaceutical properties in tablet recipes a high proportion of excipient (about 30-40%) is usually necessary, which leads to relatively large tablets and also to an increase in the production costs. Usually, a time-consuming and expensive moist granulation is necessary.

Numerous demands are made on pharmaceutical preparations, such as, for example, tablets, coated tablets or alternatively preparations in capsules, on the part of the manufacturer, the patients, but also the cost bearer in the health service:
In order to facilitate taking by the patient and thus to increase acceptance by the patient (=patient compliance), tablets should be as small as possible. This means that an optimum tablet recipe should contain a proportion of active compound which is as high as possible.
On the other hand, as a result of the increase in the proportion of active compound in a pharmaceutical preparation a more economical production is possible as a result of savings in excipients.
In order to be able to supply the active compound contained efficiently to the body, the preparation should be designed such that it has as high a bioavailability as possible. This means a tablet should rapidly disintegrate in the gastrointestinal tract, so that the active compound can rapidly dissolve.
Directly tabletable powders are therefore particularly desirable, since the process of moist granulation and the cost- and time-intensive drying step associated therewith can be dropped here.
In order to make possible processability without special industrial apparatus, the inactive excipients and the active compound should have pharmaceutical properties which are as ideal as possible. These are, for example: very good tableting behavior, good flow behavior, no adhesive behavior (e.g. sticking to die tools) and good dissolving behavior.

In the literature, numerous processes are described for improving the solubility or the rate of dissolution of profens, e.g. the incorporation of ibuprofen into cyclodextrin inclusion compounds (EP 274 444, EP 490 193) or the addition of surfactants (WO 99/17744 or U.S. Pat. No. 5,141,961). The tableting properties (flowability/formation of pressings of stable shape), however, are not improved by such processes. On the contrary, as a result of these admixtures the resulting molded articles are even more inconvenient for the patient to swallow on account of the size owing to high proportions of excipient. For example, most ibuprofen-cyclodextrin inclusion compounds are complexes in the ratio 1:1 (EP 274 44); a described ibuprofen-poloxamer complex consists of a 4:6 mixture (WO 99/17744). In a therapeutically customary dose of 200-400 mg of ibuprofen, tablets result by this means which can only be swallowed with extreme difficulty. In the therapy of rheumatic disorders, even doses of 800 mg of ibuprofen are customary. Tablets of 1.6-2.0 g, however, are no longer swallowable on account of their size.

Improvements in the rate of dissolution/solubility can be achieved by crystallization of ibuprofen from different solvents (V. Labhasetwar et al., Studies on some crystalline forms of Ibuprofen, Drug Dev. Ind. Pharm. 19(6), 631-641 (1993)). These are different crystal forms, polymorphic forms of ibuprofen being reported because of the different melting points and IR spectra. The ibuprofen prepared according to the invention is not a polymorphic form (identical melting point and identical X-ray diffractograms as the present commercial product). By means of the process according to the invention, improvements in the substance properties can moreover be obtained which have not been achieved using the processes known hitherto.

A further reference confirms the better compressibility of ibuprofen, if Eudragits® (methacrylic polymers) are present in the crystallization medium in a displacement precipitation as a result of additions. The compressibility and the flow behavior compared with the starting product are markedly improved. By means of crystallization or precipitation by a displacement precipitation using different solvents, however, ibuprofen containing intercollated Eudragits® (spherical crystal agglomerates) was produced (precipitation of the Eudragits® on account of their insolubility under the conditions), so that here too a preformulated preparation and not pure ibuprofen is already present (K. Kachrimanis et al., Int. J. Pharm. 173 (1998) 61-74, J. Pharm. Sci. 89(2) (2000) 250-259, S. T. P. Pharm. Sci. 10(5) 387-393 (2000)). The preparations thus produced have a delayed release.

J. M. E. Bunyan et al., (Solvent effects on the Morphology of Ibuprofen, AIChe Symp. Ser. 87 (1991) 44-57) investigated the influences of various solvents on the morphology of ibuprofen. Using the generally known crystallization processes, it was possible to obtain an ibuprofen having increased bulk density and better compressibility. A marked increase in the rate of dissolution, however, was not achieved. The rate of dissolution hardly differs from commercially available ibuprofen and corresponds to the profile “displacement precipitation” shown in table 1. For this also see the presentation of the rate of dissolution in table 1. In this process seed crystals are generally employed, it not being clear, however, which crystal modification these seed crystals have.

U.S. Pat. No. 4,476,248 discloses the crystallization of ibuprofen with the aim of crystallizing cubic to spherical crystals having a relatively large crystal size and high bulk density. A cooling crystallization from alcoholic solution without addition of additives is described. A marked increase in the rate of dissolution is not obtained.

It is an object of the present invention to prepare rapidly soluble, readily flowable, readily compressible and tabletable profens of high purity, which can be compressed directly to give tablets having good pharmaceutical properties in high proportions of active compound in mixtures with only low proportions of customary pharmaceutical excipients without prior granulation.

We have found that this object is achieved according to the invention by a process for the formation of profen solids, which comprises carrying out the formation of solids in the presence of one or more additives.

It is likewise an object of the present invention to use the profens crystallized in this way for the production of pharmaceutical administration forms.

The process according to the invention yields pure profen which
complies with the purity criteria of the leading and recognized pharmacopeia worldwide
is free-flowing
exhibits an easy compressibility to give tablets
on compression has no sticking properties to the tablet die
in the production of pharmaceutically customary tablets, has to be mixed with only extremely small amounts of nonactive pharmaceutical excipients
does not, as in the case of profen, customarily have to be subjected to a granulation process and dry- or moist-granulated before tableting
can thus be employed in “direct tableting processes” during tablet production
dissolves rapidly as the pure substance and from pharmaceutical formulations with only small proportions of pharmaceutical excipients in a manner not known hitherto (table 1 and table 4).

The tabletability can be markedly improved by the use of the profens prepared according to the invention. Merely by physical admixture of small percentages (below 10%) of customary pharmaceutical excipients, without further process steps tablets can be directly pressed whose physical properties such as press force/hardness ratio, friability, proportions of active compound and release rate of the active compound are markedly superior to the tablets known hitherto and the tablets described in the literature.

This is achieved by addition of water-soluble and/or water-insoluble additives during the crystallization. The choice of suitable solvents or solvent combinations also plays a crucial role in this. The addition of additives on their own or in combination with suitable solvents leads to an unexpected and surprisingly markedly positive influence both on the rate of dissolution and the flow and tableting properties. The additives added are no longer contained in the final product after formation of solids and separation have taken place or can be removed almost completely using simple washing processes. The additive accordingly causes the development of a specific crystal habit having a specific surface area, which decisively influences the substance properties. By alteration of crystal crop/crystal habit or the crystal surface area, improvements in the critical substance properties are achieved, the increase in the rate of dissolution and the improvement in the flow and tableting properties being to the fore. This process leads to a profen raw material which is suitable for direct tableting on simple physical admixture of only small proportions of pharmaceutical excipients (below 10%) without granulation.

On account of the unfavorable physicochemical properties of the profens, the demands mentioned are usually not achievable in a conventional way. Owing to these unfavorable substance properties, a more cost-intensive preparation process must be chosen—and even by means of this a biopharmaceutically optimum administration form is usually not realizable. When processing poorly soluble pharmaceuticals, high proportions of excipients, such as disintegrants, usually have to be employed. In order to improve the tabletability, as a rule higher proportions of binders, flow regulators and mold-release agents are necessary.

It was found that the physicochemical properties of the profens can be influenced, not only by changes in the habit—as described above—but moreover positively, by the formation of solids with additives. Thus in the production of tablets from ibuprofen produced according to the invention, for example, the addition of flow regulators, such as, for example, of highly disperse silicic acid (Aerosil® 200) can also largely be dispensed with. Such tablets also need only small amounts of mold-release agents such as, for example, magnesium stearate or talc during tableting. Because of the advantageous great hardnesses of these tablets, the proportions of tablet binding agents are only very low, or they can readily be dispensed with.

Thus an innovative method has been found to optimize the critical substance properties of the profen raw materials without a high proportion of excipient being contained in the final product. The novel profen is particularly suitable for the production of solid administration forms, such as tablets, which contain a proportion of active compound of 80 to 98%, preferably 90 to 98%. However, it can also be filled directly into capsules without further processing because of its good flow behavior and rapid rate of dissolution.

If needed, further active compounds can be added in the required concentration to a (tablet) recipe produced using the habit-/surface-modified profen presented here.

“Preparation” does not denote chemical synthesis here, but the steps following this of solids production and their recovery, modification and purification.

“Tableting” means the compression of the “tableting mixture” (=active compound+excipients) on a tablet press (eccentric or rotary press). In “direct tableting”, no granulation step takes place during production of the tableting mixture (neither moist granulation nor compaction). The tableting mixture is accordingly produced by simple mixing of the constituents (if appropriate after prior sieving).

The designation of the substance group consisting of the “profens” denotes active compounds containing the following structural element:

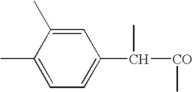

Representatives of this substance group are, for example, ibuprofen, naproxen, flurbiprofen, ketoprofen, flunoxaprofen, ibufenac, ibuproxam, pirprofen and loxoprofen, and their hydrates, solvates and physiologically tolerable salts. The invention also relates to the optically active forms, the racemates and the diastereomer mixtures of these compounds. Preferably, the process according to the invention for the formation of ibuprofen crystals is employed.

Examples of physiologically utilizable salts are salts with amino acids, e.g. lysine. Further examples of such salts are alkali metal, alkaline earth metal, ammonium and alkylammonium salts.

Pure enantiomers of the profens are obtained either by resolution (via salt formation with optically active bases) or by employing optically active starting substances in the synthesis.

The term “pharmaceutical administration form” denotes tablets, coated tablets (film-coated, lacquer-coated and sugar-coated tablets) and capsules (filled with powder, granules or pellets). In this connection, the expression “pharmaceutical administration form” does not relate exclusively to the final product, but likewise to parts or intermediates of one, such as, for example, a layer or multilayer tablet, parts of a capsule filling and the like.

Formation of solids is understood as meaning, for example, the production of crystals by displacement precipitation, crystallization by cooling the solution (cooling crystallization), evaporative crystallization or alternatively spray drying.

The designation displacement precipitation describes a process in which the formation of solids of the active compound from a solution are produced by addition of a nonsolvent. In this connection, the lowering of the temperature or the evaporation of solvent is additionally possible. The precipitated active compound is recovered by filtration and, if appropriate, by washing with a nonsolvent and subsequent drying.

During preparation of the crystals by cooling crystallization, the substance properties can be positively influenced by choice of a suitable solvent (preferably organic solvents, such as, for example, alcohols, e.g. isopropanol, if appropriate in a certain mixing ratio with, for example, water). The designation cooling crystallization describes a process in which the crystals of the active compound are produced from a solution in the solvent by lowering the temperature. The active compound produced is recovered by filtration, washing, if possible, with a nonsolvent, filtration and subsequent drying.

A further route for crystallization is evaporative crystallization, in which the solvent is removed by vaporization or evaporation.

A combination of displacement precipitation, cooling crystallization or evaporative crystallization is moreover possible.

The designation “solvent” in this connection describes a liquid in which the active compound adequately dissolves, that is, for example, ethanol, methanol, propanol, isopropanol, acetone or acetonitrile.

The designation “nonsolvent” describes a liquid in which the active compound has only low solubility, such as, for example, long-chain alcohols, but also water. The liquid thus serves as a precipitating agent.

During preparation of the crystals by displacement precipitation, the substance properties can be positively influenced by choice of suitable solvents (preferably organic solvents, such as, for example, alcohols, e.g. 1-propanol, 2-propanol, 1-butanol, 2-butanol, isobutanol, ethanol, methanol or acetone, acetonitrile, propylene glycol, glycerol or DMF) and nonsolvents (such as, for example, water, aqueous solutions of acids or organic solvents).

Preferably, those organic solvents are employed which form a miscibility gap over a certain concentration range with the nonsolvent in the presence of profens.

According to the novel process, profen crystallizate is formed by firstly dissolving profen in a suitable solvent with addition of additive. Subsequently, the solvent is reduced, for example, by lowering the temperature (cooling crystallization), by evaporation of the solvent (evaporation crystallization) or by addition of a suitable nonsolvent and, if appropriate, of a second additive dissolved therein (displacement precipitation). A particularly readily flowable and tabletable and rapidly soluble solid results if solvent and nonsolvent form a miscibility gap over a certain concentration range in the presence of the profen and if the resulting crystals is given adequate time for growth. Preferably, the formation of solids is carried out by displacement precipitation.

The formation of solids can be carried out either batchwise or continuously by cooling crystallization and/or evaporative crystallization. The formation of solids by addition of a nonsolvent (displacement precipitation) is preferably carried out as a semi-batch process, the profen being introduced in the solvent and the nonsolvent being metered in. By means of a suitable stirrer, a shear field which is as homogeneous as possible is produced using sufficiently high shearing (specific stirring power in the range from 0.2 to 2 W/kg, preferably 0.5 to 1.5 W/kg). For this, multistage stirrers and/or stirrers without sharp edges (for example impeller stirrers) can be employed. A combination of various types of stirrer is also sensible (for example an impeller stirrer in combination with axially transporting stirrer stages). The choice of an adequately long metering time for the nonsolvent is sensible (metering time between 30 min and 300 min, preferably between 40 and 210 min). The temperature is as a rule chosen in the range from 10° C. to 80° C., preferably in the range from 15° C. to 60° C., depending on the solvent. During the displacement precipitation, the solution or suspension can simultaneously be cooled or some of the solvent can be evaporated.

As mentioned above, an improvement in the substance properties, inter alia, is achieved in the presence of additives during the process of the formation of solids.

Suitable additives according to the invention are, for example, the following surfactants
partial fatty acid esters of polyoxyethylene sorbitan, such as, for example, polyethylene glycol(20)sorbitan monolaurate, monopalmitate, monostearate, monooleate; polyethylene glycol(20)sorbitan tristearate and trioleate; polyoxyethylene(5)sorbitan monooleate; polyoxyethylene(4)sorbitan monolaurate (also denoted as polysorbate)
polyoxyethylene fatty alcohol ethers, such as, for example, polyoxyethylene(4)lauryl ether, polyoxyethylene(23)lauryl ether, polyoxyethylene(10)cetyl ether, polyoxyethylene(20)cetyl ether, polyoxyethylene(10)stearyl ether, polyoxyethylene(20)stearyl ether, polyoxyethylene(10)oleyl ether, polyoxyethylene(20)oleyl ether (also denoted as macrogol fatty acid ether)
polyoxyethylene fatty acid esters, such as, for example, polyoxyethylene stearate
ethoxylated triglycerides, such as polyoxyethylene glycerol fatty acid esters, such as, for example, polyoxyethylene glycerol monoisostearate,
polyoxypropylene-polyoxyethylene block polymers (poloxamers)
suger esters (such as, for example, sucrose monolaurate, sucrose monopalmitate, sucrose monostearate, sucrose monomyristate, sucrose monooleate)
sugar ethers
alkali metal soaps (fatty acid salts), such as, for example, sodium laurate, palmitate, stearate, oleate
ionic and zwitterionic surfactants, e.g. betaines, such as, for example, cocobetaine
phospholipids

The surfactants without a PEG chain in this case have particular importance, such as especially the sugar esters and the fatty acid salts sucrose monolaurate being particularly preferably employed.

In order to achieve a removal from the final product which is as quantitative as possible, the HLB of the surfactants employed should be >8 with water as a nonsolvent, since in the case of the more lipophilic surfactants a higher proportion of surfactant can remain in the final product, which leads to increased agglomeration. The observation that surfactants which are present only during the preparation of the pharmaceutical, but are then for the most part removed by washing, permanently alter the pharmaceutical properties of the active compound is particularly surprising. An accelerated release by surfactants—in the case of their presence in the final product—is likely. Profen prepared according to the process presented here contains, however, virtually no surfacant. Surprisingly, however, it was nevertheless possible to detect an increase in the release rate due to the novel process for the formation of solids. The formation of a readily flowable product on addition of surfactants is also surprising, since surfactants actually lead to an agglutination of the crystals—if they are contained in the final product. The addition of surfactants has a decisive influence on the process of crystal formation and thus on habit and surface area of the resulting product.

Possible additives are furthermore nonsurfactants. These are, for example, the following:
sugars such as, for example, trehalose
dextrans (such as, for example, dextran 20, 60, 200)
polyvinyl alcohol, PVA
polyvinyl alcohol-polyethylene glycol graft copolymer (e.g. Kollicoat® IR)
polyvinylpyrrolidone, povidone, PVP
hydroxyethyl starch, HES (such as, for example, HES 130, 400)
cellulose ethers such as, for example, hydroxypropylcellulose HPC or hydroxyethylcellulose, HEC

The additives can be dissolved or emulsified in the solvent or in the nonsolvent.

Even on addition of one of these additives, a marked increase in the release rate (individual examples cf. table 1 and table 2) and an improvement in the flowability can be detected. The tabletability is also improved; pressings which are more stable in shape are formed. Sticking to the die tools can no longer be observed. An improvement in the flow and tableting properties can particularly be detected when using sugar esters, fatty acid salts and the nonsurfactants.

Thus nearly all critical substance properties of the profens can be positively influenced by means of the preparation process according to the invention—and the pharmaceutical further processing can thus be significantly simplified, the rate of dissolution and as a consequence of this also the bioavailability can be improved. The profens produced by the process according to the invention have an in vitro release within 5 minutes (phosphate buffer pH 7.4 according to USP XXIV by means of the paddle process at 100 rpm) of ≧70%, preferably of ≧90%.

TABLE 1
Release rate of ibuprofen prepared by displacement
precipitation (isopropanol/water)

| | Amount released [%] | | |
|---|---|---|---|
| Time | Commercial | Solvent change without | Additive: |
| [min] | article | addition | sucrose monolaurate |
| 0 | 0.0 | 0.0 | 0.0 |
| 2 | 15.1 | 20.9 | 85.2 |
| 5 | 36.9 | 48.9 | 98.8 |
| 8 | 59.5 | 67.2 | 100.0 |
| 10 | 73.9 | 76.5 | 100.0 |
| 15 | 98.0 | 94.3 | 100.0 |
| 20 | 99.0 | 100.0 | 100.0 |

TABLE 2
Release rate of ibuprofen prepared by cooling
crystallization (isopropanol)

Amount released [%]

| | Commercial article | Additive: sucrose monolaurate |
|---|---|---|
| 0 | 0.0 | 0.0 |
| 2 | 15.1 | 85.9 |
| 5 | 36.9 | 98.4 |
| 8 | 59.5 | 99.5 |
| 10 | 73.9 | 99.9 |
| 15 | 98.0 | 100.7 |
| 20 | 99.0 | 100.6 |

A further increase in the positive effects on the physicochemical properties of the active compound can be achieved by combination of a number of additives. In this connection, both a number of surfactants and a number of nonsurfactants and combinations thereof can be employed, where preferably the combination of an additive from the group consisting of the surfactants with an additive from the group consisting of the nonsurfactants, particularly preferably the combination of sugar esters/nonsurfactants, leads to a considerable increase in the rate of dissolution. Preferably, a combination of sucrose monolaurate with dextran 200, trehalose, Kollicoat® IR (=polyethylene glycol/polyvinyl alcohol graft polymer), hydroxyethyl starch, Povidon® or hydroxypropylcellulose or a combination of Tween®80 with, for example, dextran 200 is employed. The profens crystallized by the process according to the invention have an in vitro release (phosphate buffer pH 7.4; USP XXIV) of ≧70%, preferably of ≧90% (table 3).

TABLE 3
Crystallization by displacement precipitation
(additives: combination of sugar esters + nonsurfactants)

| | Amount released [%] after 2 min |
|---|---|
| Commercial article | 15.1 |
| Sucrose monolaurate | 85.2 |
| Suc. monolaurate + dextran 200 | 100.4 |
| Suc. monolaurate + trehalose | 100.1 |
| Suc. monolaurate + HPC | 100.8 |
| Suc. monolaurate + Kollicoat ® | 97.8 |
| IR | |
| Suc. monolaurate + Klucel ® LF | 98.9 |

Using the active compound having modified pharmaceutical properties (to be attributed to modifications in the surface and habit) prepared by the process described here, powder mixtures for direct tableting having a proportion of active compound of >90% can be prepared.

An example of a recipe is mentioned below which can be tabletted by the direct tableting route without the aid of further auxiliary techniques:
active compound (>90%)

The mean particle size of the profen employed does not play a crucial role; preferably it should have a mean particle size of 10 to 100 μm.
Dry binder (about 4%), such as, for example, microcrystalline cellulose (Avicel®)
Disintegration aid (about 4%), such as, for example, crosslinked sodium carboxymethylcellulose (AcDiSol®), starch derivatives, crosslinked PVP
Flow regulators (0.2 to 0.5%), such as, for example, highly disperse silica (Aerosil®). In most cases, an additional flow regulator can be dispensed with because of the good flow properties.
Lubricant (0.1 to 0.5%), such as, for example, magnesium stearate, calcium stearate, stearic acid, derivatives of stearic acid (e.g. Precirol®), talc, higher molecular weight polyethylene glycols. On account of the low adhesiveness of the profen prepared by this process, the proportion of lubricants compared with conventional recipes can be markedly lowered and serves primarily for lubricating the tablet press.

The proportions of excipient mentioned here relate to the part of the administration form which contains the active compound. An optionally additionally applied coating, which usually serves to conceal the taste of the very bitter active compound, is not taken into account.

One or more further active compounds can also be added to the pharmaceutical administration forms.

These active compounds can be, for example: pseudoephedrine, ephedrine, phenylpropanolamine, tripolidine, acetylcysteine, ambroxol, azelaic acid, dehydrocodeine, hydrocodone or coffeine. Salts of these compounds are preferred, provided the active compound is not present as a solid crystal form.

The proportion of the other active compound(s) in the pharmaceutical administration form can be between 0.5 and 70% of the proportion in % by weight of the profen, depending on the potency of the active compound and the desired effect.

The following examples are intended to illustrate the invention in greater detail, but without restricting it to these examples. The measurements of the release rate was carried out according to USP XXIV.

### EXAMPLES

1. 80 g of ibuprofen are dissolved in 100 ml of isopropanol at 40° C. Precipitation is then carried out by addition of ice water (450 ml/stirrer speed 50 rpm) during the course of 70 min. During this process cooling to 10° C. takes place. The crystals are recovered by filtration and dried in vacuo. A fine, relatively loose, readily flowable product is formed, which is prone neither to adhesion nor to cohesion. On determination of the powder dissolution (pure active compound), a 100% release is seen after 15 minutes (in phosphate buffer pH 7.4 USP XXIV), which corresponds to the commercial article Ibuprofen50 BASF AG obtainable at present. A significant increase in the release rate does not take place.
2. 5 g of naproxen are dissolved in 100 ml of isopropanol at 40° C. 3.2 g of sucrose monolaurate are added as an additive. Precipitation is then carried out by addition of ice water (450 ml/stirrer speed 200 rpm) during the course of 70 min. During this process cooling to 10° C. takes place. The crystals are recovered by filtration, washed with ice water (3×50 ml) and dried in vacuo. A relatively loose product is formed. On determination of the powder dissolution (pure active compound), a 100% release is seen after 15 seconds (in phosphate buffer pH 7.4 USP XXIV). The commercial article obtainable at present dissolves only to 35% after 2 minutes under identical conditions; a 100% dissolution is only achieved after >30 min.
3. 5 g of naproxen are dissolved in 100 ml of isopropanol at 40° C. 8 g of Tween®80 are added as an additive. Precipitation is then carried out by addition of ice water (450 ml/stirrer speed 200 rpm) during the course of 70 min. During this process cooling to 10° C. takes place. The crystals are recovered by filtration, washed with ice water (3×50 ml) and dried in vacuo. A relatively loose product is formed. On determination of the powder dissolution (pure active compound), a 100% release is seen after 15 seconds (in phosphate buffer pH 7.4 USP XXIV). The commercial article obtainable at present dissolves only to 35% after 2 minutes under identical conditions; a 100% dissolution is only achieved after >30 min.
4. 80 g of ibuprofen are dissolved in 100 ml of isopropanol at 40° C. 3 g of sucrose monolaurate are added as an additive. Precipitation is then carried out by addition of ice water (450 ml/stirrer speed 200 rpm) during the course of 70 min; during this process, cooling to 10° C. takes place; the crystals are recovered by filtration, washed with ice water (3×150 ml) and dried in vacuo. A fine, relatively loose, readily flowable product is formed, which is prone neither to adhesion nor to cohesion. On determination of the powder dissolution (pure active compound) a 100% release is seen after 5 minutes (85% within 2 minutes) (in phosphate buffer pH 7.4 USP XXIV). The commercial article obtainable at present dissolves only to <20% after 2 minutes under identical conditions; a 100% dissolution is only achieved after >15 min.

5. 80 g of ibuprofen are dissolved in 100 ml of isopropanol at 40° C. 3 g of sucrose monolaurate are added as an additive. Precipitation is then carried out by addition of ice water (450 ml/stirrer speed 200 rpm) during the course of 70 min; during this process, cooling to 10° C. takes place. The crystals are recovered by filtration, washed with ice water (3×150 ml) and dried in vacuo. 3 g of the product are washed again with water (10×50 ml). A fine, relatively loose, readily flowable product is formed, which is prone neither to adhesion nor to cohesion. On determination of the powder dissolution (pure active compound), a 100% release is seen after 5 minutes (85% up to minute 2) (in phosphate buffer pH 7.4 USP XXIV). The commercial article obtainable at present dissolves only to <20% after 2 minutes under identical conditions; a 100% dissolution is only achieved after >15 min.
6. 80 g of ibuprofen are dissolved in 100 ml of isopropanol at 40° C. 1.0 g of sucrose monolaurate is added as an additive. Precipitation is then carried out by addition of ice water (450 ml/stirrer speed 200 rpm) during the course of 70 min; during this process, cooling to 10° C. takes place. The crystals are recovered by filtration, washed with ice water (3×150 ml) and dried in vacuo. A fine, relatively loose, readily flowable product is formed, which is prone neither to adhesion nor to cohesion. On determination of the powder dissolution (pure active compound) a 100% release is seen after 5 minutes (85% within 2 minutes) (in phosphate buffer pH 7.4 USP XXIV).
7. 45 g of ibuprofen are dissolved in 100 ml of isopropanol at 20° C. 1.5 g of sucrose monolaurate are added as an additive. Precipitation is then carried out by addition of water (450 ml/stirrer speed 200 rpm) during the course of 70 min. The crystals are recovered by filtration, washed with ice water (3×150 ml) and dried in vacuo. A fine, relatively loose, readily flowable product is formed, which is prone neither to adhesion nor to cohesion. On determination of the powder dissolution (pure active compound) a 100% release is seen after 5 minutes (85% within 2 minutes) (in phosphate buffer pH 7.4 USP XXIV).
8. 80 g of ibuprofen are dissolved in 100 ml of isopropanol at 40° C. 3 g of sucrose monolaurate are added as an additive. Precipitation is then carried out by addition of ice water (450 ml), to which 8 g of dextran 200 are added (70 min). During this process, cooling to 10° C. takes place. The crystals are recovered by filtration, washed with ice water (3×150 ml) and dried in vacuo. A fine, relatively loose, readily flowable product is formed, which is prone neither to adhesion nor to cohesion. On determination of the powder dissolution (pure active compound) a 100% release is seen after <30 seconds (in phosphate buffer pH 7.4 USP XXIV). The commercial article obtainable at present dissolves only to <20% after 2 minutes under identical conditions; a 100% dissolution is only achieved after >15 min.
9. 80 g of ibuprofen are dissolved in 100 ml of isopropanol at 40° C. Precipitation is then carried out by addition of ice water (450 ml/stirrer speed 200 rpm) during the course of 70 min. 3 g of sucrose monolaurate and 8 g of trehalose are employed as additives. During this process, cooling to 10° C. takes place. The crystals are recovered by filtration, washed with ice water (3×150 ml) and dried in vacuo. A fine, relatively loose, readily flowable product is formed, which is prone neither to adhesion nor to cohesion. On determination of the powder dissolution (pure active compound) a 100% release is seen after <30 seconds (in phosphate buffer pH 7.4 USP XXIV). The commercial article obtainable at present dissolves only to <20% after 2 minutes under identical conditions; a 100% dissolution is only achieved after >15 min.
10. 80 g of ibuprofen are dissolved in 100 ml of isopropanol at 40° C. 3 g of sucrose monolaurate are added as additives. Precipitation is then carried out by addition of ice water with 8 g of hydroxypropylcellulose (Klucel®LF) (450 ml/stirrer speed 200 rpm) during the course of 70 min; during this process, cooling to 10° C. takes place. The crystals are recovered by filtration, washed with ice water (3×150 ml) and dried in vacuo. A fine, relatively loose, readily flowable product is formed, which is prone neither to adhesion nor to cohesion. On determination of the powder dissolution (pure active compound) a 100% release is seen after <30 seconds (in phosphate buffer pH 7.4 USP XXIV). The commercial article obtainable at this time dissolves only to <20% after 2 minutes under identical conditions; a 100% dissolution is only achieved after >15 min.
11. In a stirring vessel operated batchwise, ibuprofen was precipitated on the 3 l scale from 2-propanol with water using sucrose monolaurate and Klucel LF. A double-walled glass container having three flow disrupters and a beveled blade turbine was employed as the stirring element. A specific stirring power of 0.25 W/kg was introduced. 411 g of ibuprofen were introduced and dissolved in 936 g of a solution of 2-propanol and sucrose monolaurate (1.0% by weight of sucrose monolaurate in the solution). 3754 g of a 0.24% by weight water/Klucel LF solution were metered in at 20° C. in the course of 10 min. The solid was separated off by means of a suction filter and washed with water. A strongly agglomerated crystallizate results, which is cohesive (poor flowability). The solid nevertheless has a comparatively good rate of dissolution. After 5 and 8 min respectively, 81 and 90% respectively of the active compound are dissolved.
12. In a stirring vessel operated batchwise, ibuprofen was precipitated on the 3 l scale from 2-propanol with water using sucrose monolaurate and Klucel LF. A double-walled glass container having three flow disrupters and a beveled blade turbine was employed as the stirring element. A specific stirring power of 1 W/kg was introduced. 414 g of ibuprofen were introduced and dissolved in 943 g of a solution of 2-propanol and sucrose monolaurate (1.0% by weight of sucrose monolaurate in the solution). 3780 g of a 0.24% by weight water/Kollicoat® IR solution were metered in at 20° C. in the course of 70 min. The solid was separated off by means of a suction filter and washed with water. The crystallizate is very readily flowable and has a good rate of dissolution. After 5 and 8 min respectively, 87 and 95% respectively of the active compound are dissolved.
13. In a 3 l stirring vessel, 357 g of ibuprofen were introduced and dissolved in 643 g of a solution of 2-propanol and sucrose monolaurate (1.2% by weight of sucrose monolaurate in the solution). The stirring element used was a beveled blade turbine. The specific power input by the stirrer was 1 W/kg. 3314g of a 0.97% by weight water/Kollicoat® IR solution were metered in at 20° C. in the course of 70 min. The solid was separated off by means of a suction filter and washed with water. The crystallizate is very readily flowable and has a good rate of dissolution. After 5 and 8 min respectively, 92 and 100% respectively of the active compound are dissolved.
14. In a 3 l stirring vessel, 357 g of ibuprofen were introduced and dissolved in 643 g of a solution of 2-propanol and sucrose monolaurate (1.2% by weight of sucrose monolaurate in the solution). The stirring element used was a beveled blade turbine (specific power input: 1 W/kg). 3314g of a 0.97% by weight water/Kollicoat® IR solution were metered in at 20° C. in the course of 120 min. The solid was separated off by means of a suction filter and washed with water. The crystallizate is very readily flowable and has a good rate of dissolution.
15. In a 3 l stirring vessel, 357 g of ibuprofen were introduced and dissolved in 643 g of a solution of 2-propanol and sucrose monolaurate (1.2% by weight of sucrose monolaurate in the solution). The stirring element used was an impeller stirrer (specific power input: 1 W/kg). 3314g of a 0.97% by weight water/Kollicoat® IR solution were metered in at 20° C. in the course of 120 min. The solid was separated off by means of a suction filter and washed with water. The crystallizate is very readily flowable and has a good rate of dissolution.
16. In a 3 l stirring vessel, 357 g of ibuprofen were introduced and dissolved in 643 g of a solution of 2-propanol and sucrose monolaurate (1.2% by weight of sucrose monolaurate in the solution). The stirring element used was an impeller stirrer (specific power input: 0.15 W/kg). 3314g of a 0.97% by weight water/Kollicoat® IR solution were metered in at 20° C. in the course of 120 min. The solid was separated off by means of a suction filter and washed with water. The crystallizate is more strongly agglomerated than the solid from example 6.

17. The suitability for direct tableting is illustrated by the following example: 80 g of ibuprofen are dissolved in 100 ml of isopropanol at 40° C. 3 g of sucrose monolaurate are added as an additive. Precipitation is then carried out by addition of ice water (450 ml/stirrer speed 200 rpm) during the course of 70 min; during this process cooling to 10° C. takes place. The crystals are recovered by filtration, washed with ice water (3×150 ml) and dried in vacuo. A fine, relatively loose, readily flowable product is formed, which is prone neither to adhesion nor to cohesion. A powder mixture for direct tableting results with the following excipients:

| | % by weight |
|---|---|
| Ibuprofen | 91.20 |
| Avicel PH102 | 4.00 |
| AcDiSol | 4.00 |
| Aerosil | 0.50 |
| Mg stearate | 0.30 |

The tablets pressed by direct tableting fulfill the requirements of Ph. Eur.; the maximum deviation on determination of the homogeneity of the material is 0.9%. The tablet surface is uniform. The ibuprofen prepared according to the invention is thus suitable for direct tableting (with a high active compound content of >90%). On account of the good flow properties of the active compound, the proportion of Aerosil® can be lowered further. A reduction in the proportion of lubricant (magnesium stearate) is also possible.

On determination of the release behavior, a 100% release is seen after 2 minutes (including the disintegration time of <30 sec) (in phosphate buffer pH 7.4 USP XXIV).

Incorporation of the ibuprofen commercial article obtainable at present into the abovementioned tableting mixture produces tablets having severe surface defects, since strong adhesion (sticking) to the die tools takes place. This ibuprofen is not suitable for direct tableting (at a high active compound content of >90%). On determination of the release behavior a 100% release is seen after 10 minutes (including the disintegration time of <30 sec) (in phosphate buffer pH 7.4 USP XXIV). In table 4, the release rates of tablets (ibuprofen commercial article/ibuprofen solvent change with sucrose monolaurate (4%, based on ibuprofen)/ibuprofen solvent change with sucrose monolaurate and dextran 200 (4 or 10%, based on ibuprofen)) are shown comparatively.

TABLE 4
Overview of the release rate from tablet formulations

| | Amount released [%] | | |
|---|---|---|---|
| | | | Displacement |
| | | Displacement | Precipitation with |
| | | precipitation | additive: |
| | Commercial | with additive: | suc. monolaurate + |
| Time | article | sucrose monolaurate | dextran 200 |
| Minute 2 | 41.3 | 79.5 | 92.8 |
| Minute 5 | 69.3 | 99.7 | 100.6 |
| Minute 10 | 100.1 | 100.8 | 100.1 |

18. 80 g of ibuprofen are dissolved in 100 ml of 90% strength isopropanol (10% double-distilled water) at 40° C. 1.2 g of sucrose monolaurate are added as an additive. Crystallization is then initiated by cooling. To this end, the temperature is lowered to 15° C. in the course of 150 min and then to 0° C. in the course of 12 h. During this process, stirring is carried out at a stirrer speed of 50 rpm. The crystals are recovered by filtration, dried in vacuo and then washed with deagglomeration using ice water (3×150 ml) and dried again in vacuo. A fine, relatively loose, readily flowable product is formed, which is prone neither to adhesion nor to cohesion. On determination of the powder dissolution (pure active compound) a 100% release is seen after approximately 5 min (in phosphate buffer pH 7.4 USP XXIV). The commercial article obtainable at present dissolves only to <20% after 2 minutes under identical conditions; a 100% dissolution is achieved only after >15 min.

1. A process for the formation of ibuprofen solids wherein one or more additives are used in the solids formation process, wherein the ibuprofen is dissolved in a suitable solvent while additives are added, wherein the additions used are a sugar ester, sugar, dextrans, povidone, polyvinyl alcohol-polyethylene glycol graft copolymers or combinations thereof and the additives used are after solids formation and removal have taken place no longer present in the end product or can be removed by washing, wherein the average size of the ibuprofen particles is in the range from 10 to 100 μm.

2. A process as claimed in claim 1, wherein the formation of solids is carried out by displacement precipitation.

3. A process as claimed in claim 2, wherein the process is carried out as a semibatch process.

4. A process as claimed in claim 1, wherein the formation of solids is carried out as a cooling crystallization.

5. A process as claimed in claim 1, wherein the formation of solids is carried out by combination of a displacement precipitation with a cooling crystallization.

6. A process as claimed in one of claims 1 to 5, wherein, as additives, a combination of sucrose monolaurate with dextran 200, Trehalose, Povidon or a polyvinyl alcohol-polyoxyethylene graft copolymer is employed.

7. A process as claimed in either of claims 2 or 3, wherein the solvent used and the nonsolvent form a miscibility gap over one part of the concentration range in the presence of profen.

8. A process as claimed in one of claims 1 to 7, wherein at least one stirrer having a specific stirring power of 0.2 to 2 W/kg is employed in the process.

9. A process as claimed in one of claims 2, 3 or 7, wherein the metering time for the nonsolvent is between 30 and 300 min.
